(19) United States
(12) Patent Application Publication
(10) Pub. No.: US 2006/0246409 A1
(43) Pub. Date: Nov. 2, 2006

(54) ScreenXbook publishing method

(76) Inventor: Aram Akopian, Glasgow (GB)

Correspondence Address: Dr. ARAM AKOPIAN, 4450, BOYCE HALL, UCR, 900 UNIVERSITY AVE., RIVERSIDE, CA, 92521, US

(21) Appl. No.: 11/099,309

(22) Filed: Apr. 6, 2005

(51) Int. Cl. A01H 5/02; G09B 5/00

(52) U.S. Cl. 434/317

(57) ABSTRACT

The invention relates to an electronic method of publishing in a novel format named “ScreenXbook”. The method comprises electronic publishing of printed material in a manner (format) that efficiently displays (“screens”) this material on a screen, for facilitated human reading.

### CROSS-REFERENCE TO A RELATED PROVISIONAL APPLICATION

This nonprovisional utility patent application is claiming the benefit of a related copending provisional application No. 60/567,529 filed with USPTO on May 4, 2004.

### BACKGROUND OF THE INVENTION

1. Field of the Invention

The invention relates generally to an electronic method of publishing. More particularly, the invention relates to electronic publishing of printed material in a novel format named “ScreenXbook”.

2. Prior Art

The available means of book or other printed material publishing, whether traditional ones on paper or electronic ones introduced so far, have not made reading easier than usual.

The only format of publishing that facilitated assimilation of certain types of printed material has been the “audio-book” format. However, in this format, the mode of assimilation has been changed from visual to auditory. The method of publishing for which this application is filed preserves the visual mode of assimilation, while facilitating the said assimilation at the same time (at least for certain types of printed material, as explained in the Detailed Description).

In all the known electronic publishing methods, the contents are displayed either on a personal computer's screen, or by a specially designed apparatus of an “electronic book”, such as those disclosed in U.S. Pat. No. 5,991,594 to Froeber and Kim, U.S. Pat. No. 5,897,324 to Tan, U.S. Pat. No. 5,761,485 to Munyan, and U.S. Pat. No. 5,534,888 to Blair et al.

The advent of personal computer, and then Internet, has popularised the phenomena of electronic publishing of printed material and reading from computer screen. Yet, the used methods of electronic publishing do not fully exploit the capabilities of existing technology. This is particularly the case for certain types of literary and other non-scientific reading material, as detailed further in this application in the best mode disclosure.

It is widely accepted that the current methods of electronic publishing of printed material are largely based on replacing paper pages with virtual equivalents displayed by electronic screen, without change in the traditional static presentation of contents on the pages. This includes the latest inventions related to electronic publishing and electronic book display (U.S. Pat. No. 5,860,073 to Schofield et al., U.S. Pat. No. 6,105,044 to Derose and Vogel, Pat. No. WO0225,477 to Pujol et al., Pat. No. FR2,829,845 to Schweitzer).

U.S. Pat. No. 5,802,533 to Walker, incorporated herein as all the other above patents by reference, describes an apparatus and a complex method attempting to improve electronic text presentation. The method comprises extracting text specific attributes from machine readable text and varying text presentation in accordance with said attributes, wherein said text specific attributes include a text difficulty measure, and said text presentation includes an automatic advancement rate, which is varied automatically in accordance with said text difficulty measure.

However, despite the relatively long presence of personal computer and Internet, up until now no method has been introduced for an improved and standartised way of electronic publishing of printed material that is not conceptually based on text specific attributes and efficiently displays this material on screen for convenient and greatly facilitated human reading.

### SUMMARY OF THE INVENTION

The purpose of the present invention is to provide a new method of electronic publishing of printed material in a format which facilitates the visual experience of reading.

The method comprises the following steps:

a) Input of printed material (i.e. textual and graphic material that can be published in the traditional way on paper) into a computer-code electronic data form which, upon access, displays it for facilitated human reading on screen, as follows:

The text part of the said printed material is displayed on screen in letters of a single light or bright color against a dark-color background, by segments stretching over 1 or 2 horizontal lines/half-lines each;

these segments appear on the screen in the same uniform appearance effect revealing the whole segment, one after the other from top to the bottom, each a number of seconds after the other, at time intervals convenient for their assimilation (and thus filling “screen pages” that follow one another).

The accompanying illustrations that form part of the said printed material are displayed on separate “screen pages”, separately from the main text part.

b) Saving of the said electronic data form into a permanent electronic record form such as file, or recording of the said electronic data form's display onto a cinematographic film or video film.

c) Publishing of the said file by displaying or distributing it on Internet (or another computer network), or by recording it on a media support such as floppy disk or CD.

Detailed descriptions of the last two steps of the method (steps b) and c)) are omitted in this application so as to not obscure the description of the invention with unnecessary details of well-known methods and devices used for accomplishment of the said steps. Information for enabling the first step of the method (step a)) is provided as embodiment examples below, and further in the application both as a detailed single embodiment description and as the best mode disclosure.

In one embodiment of the invention, the input of printed material into a computer-code electronic data form, which upon access displays it for facilitated human reading on screen as described in this invention, involves:

incorporating the text of the said material into a set of commands, written in a programming language such as C++ or VISUAL BASIC, to be saved as an executable file;

digitizing the graphic elements of the said material as files, using commercially available scanners and computer equipment;

linking the said files of the graphic elements to the said set of commands which has incorporated the text of the printed material.

In an alternative embodiment, the input of printed material into a computer-code electronic data form, which upon access displays it for facilitated human reading on screen as described in this invention, involves:

incorporating the text of the said material into a set of operations, accomplished by using a freeware limited-programming tool such as those for making animated desktop wallpapers/screensavers;

digitizing the graphic elements of the said material as files, using commercially available scanners and computer equipment;

linking the said files of the graphic elements to the said set of operations which has incorporated the text of the printed material.

In another alternative embodiment, the input of printed material into a computer-code electronic data form, which upon access displays it for facilitated human reading on screen as described in this invention, involves:

incorporating the text of the said material into a set of operations, accomplished by using a commercial software capable of making animations such as Swish, Macromedia Flash or Microsoft Powerpoint;

digitizing the graphic elements of the said material as files, using commercially available scanners and computer equipment;

linking the said files of the graphic elements to the said set of operations which has incorporated the text of the printed material.

One advantage of the invention, as mentioned above, is that it facilitates the visual experience of reading from screen, at least for certain types of literary and other non-scientific reading material, as detailed in the ensuing part of the application which covers the best mode disclosure.

Another advantage of the invention is that the said facilitation of reading from screen is achieved by electronic publishing of printed material in a standartised format, with text segmentation variations upon publication dependent only on the publisher's/author's preferences.

Yet another advantage of the invention is that the method of publishing of printed material for which this application is filed preserves the visual mode of its assimilation, while facilitating the said assimilation at the same time.

### DETAILED DESCRIPTION OF THE INVENTION

In the following description, for purposes of explanation and not limitation, specific details are set forth in order to provide a thorough understanding of the present invention.

They include the best mode disclosure and a detailed single embodiment description (method of operation thereof). It is understood, however, that the invention can be embodied in a multitude of different ways as defined and covered by the claims.

Best Mode Disclosure

The best mode of the present invention involves a number of important factors.

First, in the method of electronic publishing of this invention, the text part of printed material is displayed on screen by very few lines per “screen page” in relatively big fonts, so as to ration the presentation of information in order not to overload the reader with it, and yet so as to present as much of a complete segment of information (such as a complete sentence or paragraph) on the same screen page as possible.

In one preferred embodiment, the number of lines per screen page does not exceed 6-7, and the lines make full use of screen, except, of course, its minimal margins. The fonts are the size to display on average 35-40 characters per line, including spaces.

In an alternative preferred embodiment, the number of lines per full screen page is 10-11, but the lines are approximately half-screen in length, preferably aligned to the left margin of the screen and leaving most of the right half-screen space free. The fonts are the size to display on average 35-40 characters per line, including spaces. In this embodiment, the “screen page” is the space occupied by the text on screen, and illustrations accompanying the said text can be displayed alongside a “screen page” of text in the remaining free space on screen.

Second, in the best embodiment of this invention, the vertical line separation on a “screen page” is set at double line spacing, i.e. the lines of text are displayed on screen uniformly separated from each other by distance equivalent to the height of a single text line.

Third, in the best embodiment of this invention, the said text part of printed material is displayed in white color against black background (“screen page”).

Fourth, the segments of display of the said text part, as defined in claims, appear on the screen in the same uniform appearance effect revealing the whole segment. In the best embodiment of this invention, the segments of text are revealed in the appearance effect technically referred to as “boxing/splitting out”. In alternative embodiments, a different appearance effect could be used for revealing the said segments of text, with some appearance effects coming through much less serious and more outstanding to the eye than others.

Fifth, these said segments of text appear on the screen from top to the bottom a number of seconds each after the other, at time intervals convenient for their assimilation (thus filling “screen pages” that follow one another).

In one preferred embodiment, the said time intervals are determined and set by the publisher, according to the author's preferences/speed of reading, or, alternatively, according to the speed of reading of the said text segments by an average reader (could be determined, for example, by referring to a pre-established table assigning a time interval of assimilation by an average reader of text segments of a given character count range). In this embodiment, the screenXbook automatically displays its contents upon access, without possibility of any manual control of the said time intervals within a “screen page” by the reader.

In another preferred embodiment, the screenXbook is published in a format which allows the text segments' appearance on a “screen page” (filling of a “screen page”) to be controlled manually by the reader, via a control device such as computer mouse or its equivalent remote control. This allows the presentation of text on screen to be stopped or paused at any moment. In this embodiment, the reader can read the text segment by segment at their individual most comfortable reading speed, not having to hurry up to read the next line or wait for it a few seconds. Such embodiment of the invention is particularly useful for people who have reading difficulties, whether imposed by their poor level of education and prior reading experience, or by conditions affecting learning, such as dyslexia or old age.

Sixth, in the best embodiment of this invention, the said segments of text are defined upon input of text into electronic data form, by the publisher or the author, in such a way as to incorporate maximum of information within a segment's limits (according to these limits' definition in the claims) without ending the segment in the middle of a rigid lexical structure (as in “end of sentence. Adjective”), and at the same time keep the reader's interest in the completion of that piece of information.

Seventh, to facilitate reading of screenXbooks and in analogy to free space left in printed books to indicate passage to another paragraph, beginning of a new paragraph often requires start of a new screen page in a screenXbook, even if the previous one was not filled to completion.

Exemplary single embodiment description (method of operation thereof)

The following single embodiment description allows practical demonstration of the ScreenXbook publishing method and is intended to give a visual idea of the invention. It is exemplary and is not intended to limit in any way the scope of this invention, which is defined by claims.

The exemplary embodiment provides step-by-step instructions for the input of printed material into a computer-code electronic data form which upon access displays it for facilitated human reading on screen as described in this invention. Although many alternative ways exist for enabling such input (as described in the Summary), for purposes of minimal complexity the description uses a commercial software tool capable of making animations. For the very same purpose, use of Microsoft Powerpoint is chosen in this example over use of other software programs such as Swish, Macromedia Flash, or even the freeware alternative to Microsoft Powerpoint called Powerbullet Presenter. In step 1, upon starting Microsoft Powerpoint, version 2000, the embodiment operator, hereinafter referred to as ‘the operator’, creates a new presentation using the ‘Blank presentation’ option and any Autolayout.

In step 2, the operator selects the Page Setup option from the File menu, and sets the ‘Slides sized for’ subsection at ‘On-screen Show’; the ‘Width’ subsection at ‘10 inches’; the ‘Height’ subsection at ‘7.5 inches’; the ‘Number slides from’ subsection at ‘1’; the ‘Orientation of Slides’ subsection at ‘Landscape’; and the ‘Orientation of Notes, handouts & outline’ subsection at ‘Portrait’.

In step 3, after maximizing the program window, the operator sets the slide zoom at 75% and moves the adjacent bars more to the left and down until the whole of the slide can be comfortably seen.

In step 4, the operator selects the Select All option from the Edit menu, and then selects the Cut option from the same Edit menu.

In step 5, the operator selects the Text Box option from the Insert menu and inserts it anywhere in the blank field.

In step 6, the operator types in ‘ABC’ in capital letters and Times New Roman font of size 44.

In step 7, the operator selects the Select All option from the Edit menu.

In step 8, the operator selects the Font option from the Format menu and sets the Color parameter at White.

In step 9, the operator selects the Background option from the Format menu and, after setting it at Black, clicks on the Apply option.

In step 10, the operator spreads the text box's left border to the very left edge of the slide and the right border to the very right edge of the slide.

In step 11, the operator moves the text box so that its top border touches the top edge of the slide.

In step 12, the operator selects the box and then selects the Copy option from the Edit menu.

In step 13, the operator pastes a new box into the slide and places it right below the previous one, so that there is no space between its top border and the bottom border of the previous text box.

In step 14, the operator repeats the previous step until seven text boxes in total are made present in the slide's field.

In step 15, the operator makes the font size of the very first text box 40, and moves it to the right until its left border aligns with the bar of letter ‘B’ in the position of the text box prior to the execution of the movement.

In step 16, the operator selects the Select All option from the Edit menu and makes the font size 40.

In step 17, the operator selects the Align or Distribute option from the Draw menu and clicks on Align Right.

In step 18, the operator selects the very top text box and then selects the Cut option from the Edit menu.

In step 19, the operator selects the Custom Animation option from the Slide Show menu. In the ‘Check to animate slide objects’ section, the operator ticks all text lines in the descending order. In the ‘Order and Timing’ section's ‘Animation order’ subsection, the operator selects Text1. Then, the operator sets the ‘Start animation’ subsection on the right to ‘Automatically, 0.8 seconds after previous event’.

In step 20, in the ‘Order and Timing’ section's ‘Animation order’ subsection, the operator selects Text2. Then, the operator sets the ‘Start animation’ subsection on the right to ‘Automatically, 1.5 seconds after previous event’.

In step 21, in the ‘Order and Timing’ section's ‘Animation order’ subsection, the operator selects Text3. Then, the operator sets the ‘Start animation’ subsection on the right to ‘Automatically, 2.0 seconds after previous event’.

In step 22, in the ‘Order and Timing’ section's ‘Animation order’ subsection, the operator selects Text4. Then, the operator sets the ‘Start animation’ subsection on the right to ‘Automatically, 1.7 seconds after previous event’.

In step 23, in the ‘Order and Timing’ section's ‘Animation order’ subsection, the operator selects Text5. Then, the operator sets the ‘Start animation’ subsection on the right to ‘Automatically, 2.0 seconds after previous event’.

In step 24, in the ‘Order and Timing’ section's ‘Animation order’ subsection, the operator selects Text6. Then, the operator sets the ‘Start animation’ subsection on the right to ‘Automatically, 3.0 seconds after previous event’.

In step 25, the operator repeats the selection of Text1 to Text6 in the ‘Order and Timing’ section, and after each selection, in the ‘Effects’ section next to the ‘Order and Timing’, sets the ‘Entry animation and sound’ subsection at ‘Box’, ‘Out’, and ‘No sound’; the ‘After Animation’ subsection at ‘Don't Dim’; and the ‘Introduce text’ subsection at ‘All at once’. The operator does not tick the ‘Grouped by’ subsection.

In step 26, the operator presses OK at the top right corner of the ‘Custom Animation’ panel.

In step 27, the operator selects the Slide Sorter option from the View menu.

In step 28, the operator selects the Select All option from the Edit menu, and then selects the Duplicate option from the same Edit menu.

In step 29, the operator repeats the previous step until ten slides are made present in total.

In step 30, the operator double-clicks on the first slide to return to the full slide mode.

In step 31, the operator selects ‘ABC’ in the first very top text box, and in its stead types in the following text as it appears, i.e. case-sensitively, between the square brackets:

[This being a story—].

In step 32, instead of ‘ABC’s of the following 2 text boxes, in the same descending order, the operator types in the following 2 lines:

[and a truer one than it may appear—]

[of a mining country,].

In step 33, the operator selects the fifth text box from top and selects the Cut option from the Edit menu.

In step 34, the operator selects ‘ABC’ in the fourth text box from top, and in its stead types in [it is quite to be expected that it will be a]. Then, the operator presses Enter and types in on a new line in the same text box [hard-luck story.].

In step 35, in the fourth text box from top, the operator introduces an empty line between the first and second lines of text by pressing Enter at the beginning of the second line. Then, the operator sets the font size of the empty line at 20.

In step 36, the operator selects ‘ABC’ in the next text box and in its stead types in [But that depends on the point of view.].

In step 37, the operator passes to the next slide and instead of ‘ABC’s types in into the first top 3 text boxes, in the same descending order as given, the following lines:

[Hard luck is a mild way of terming it]

[so far as Kink Mitchell and]

[Hootchinoo Bill are concerned;].

In step 38, the operator selects the fifth text box from top and selects the Cut option from the Edit menu.

In step 39, the operator selects ‘ABC’ in the fourth text box from top, and in its stead types in [and that they have a decided opinion on]. Then, the operator presses Enter and types in on a new line in the same text box [the subject].

In step 40, in the fourth text box from top, the operator introduces an empty line between the first and second lines of text by pressing Enter at the beginning of the second line. Then, the operator sets the font size of the empty line at 20.

In step 41, the operator selects ‘ABC’ in the next text box and in its stead types in [is a matter of common knowledge in the].

In step 42, the operator passes to the next slide and instead of ‘ABC’ in the first very top text box types in [Yukon country.].

In step 43, the operator selects all the remaining text boxes and selects the Cut option from the Edit menu.

In step 44, the operator passes to the next slide and instead of ‘ABC’s types in into the first top 3 text boxes, in the same descending order as given, the following lines:

[It was in the fall of 1896]

[that the two partners came down to the]

[east bank of the Yukon,]

In step 45, the operator selects the fifth text box from top and selects the Cut option from the Edit menu.

In step 46, the operator selects ‘ABC’ in the fourth text box from top, and in its stead types in [and drew a Peterborough canoe from a]. Then, the operator presses Enter and types in on a new line in the same text box [moss-covered cache.].

In step 47, in the fourth text box from top, the operator introduces an empty line between the first and second lines of text by pressing Enter at the beginning of the second line. Then, the operator sets the font size of the empty line at 20.

In step 48, the operator selects ‘ABC’ in the next text box and in its stead types in [They were not].

In step 49, the operator passes to the next slide and instead of ‘ABC’s types in into the text boxes, in the same descending order as given, the following lines:

[particularly pleasant looking objects.]

[A summer's prospecting,]

[filled to repletion with hardship]

[and rather empty of grub,]

[had left their clothes in tatters]

[and themselves worn and cadaverous.].

In step 50, the operator selects the second text box from top and selects the Cut option from the Edit menu.

In step 51, the operator selects ‘ABC’ in the first text box from top, and in its stead types in [A nimbus of mosquitoes buzzed about]. Then, the operator presses Enter and types in on a new line in the same text box [each man's head.].

In step 52, in the first text box from top, the operator introduces an empty line between the first and second lines of text by pressing Enter at the beginning of the second line. Then, the operator sets the font size of the empty line at 20.

In step 53, instead of ‘ABC’s of the following 2 text boxes, in the same descending order, the operator types in the following 2 lines:

[Their faces were coated with blue clay.]

[Each carried a lump of this damp clay,].

In step 54, the operator selects the last text box from top and selects the Cut option from the Edit menu.

In step 55, the operator selects ‘ABC’ in the fourth text box from top, and in its stead types in [and, whenever it dried and fell from their]. Then, the operator presses Enter and types in on a new line in the same text box [faces,].

In step 56, in the fourth text box from top, the operator introduces an empty line between the first and second lines of text by pressing Enter at the beginning of the second line. Then, the operator sets the font size of the empty line at 20.

In step 57, the operator passes to the next slide and instead of ‘ABC’ in the first very top text box types in [more was daubed on in its place.].

In step 58, the operator selects the third text box from top and selects the Cut option from the Edit menu.

In step 59, the operator selects ‘ABC’ in the second text box from top, and in its stead types in [There was a querulous plaint in their]. Then, the operator presses Enter and types in on a new line in the same text box [voices,].

In step 60, in the fourth text box from top, the operator introduces an empty line between the first and second lines of text by pressing Enter at the beginning of the second line. Then, the operator sets the font size of the empty line at 20.

In step 61, instead of ‘ABC’s of the following 3 text boxes, in the same descending order, the operator types in the following 3 lines:

[an irritability of movement and gesture,]

[that told of broken sleep and a losing]

[struggle with the little winged pests.].

In step 62, the operator selects the Slide Sorter option from the View menu.

In step 63, the operator selects the last three slides among the ten and selects the Cut option from the Edit menu.

In step 64, the operator double-clicks on the first slide to return to the full slide mode.

In step 65, the operator selects the Slide Transition option from the Slide Show menu. In the panel, the operator sets the ‘Effect’ section at ‘No transition’; the ‘Advance’ section at ‘Automatically after 00:13.5’; and the ‘Sound’ section at ‘No sound’. Then, the operator clicks on the Apply option.

In step 66, the operator passes to the next slide.

In step 67, the operator selects the Custom Animation option from the Slide Show menu. In the ‘Order and Timing’ section's ‘Animation order’ subsection, the operator selects Text1. Then, the operator sets the ‘Start animation’ subsection on the right to ‘Automatically, 0 seconds after previous event’.

In step 68, in the ‘Order and Timing’ section's ‘Animation order’ subsection, the operator selects Text2. Then, the operator sets the ‘Start animation’ subsection on the right to ‘Automatically, 2.0 seconds after previous event’.

In step 69, in the ‘Order and Timing’ section's ‘Animation order’ subsection, the operator selects Text3. Then, the operator sets the ‘Start animation’ subsection on the right to ‘Automatically, 2.0 seconds after previous event’.

In step 70, in the ‘Order and Timing’ section's ‘Animation order’ subsection, the operator selects Text4. Then, the operator sets the ‘Start animation’ subsection on the right to ‘Automatically, 2.0 seconds after previous event’.

In step 71, in the ‘Order and Timing’ section's ‘Animation order’ subsection, the operator selects Text5. Then, the operator sets the ‘Start animation’ subsection on the right to ‘Automatically, 2.7 seconds after previous event’.

In step 72, the operator selects the Slide Transition option from the Slide Show menu. In the panel, the operator sets the ‘Effect’ section at ‘No transition’; the ‘Advance’ section at ‘Automatically after 00:13’; and the ‘Sound’ section at ‘No sound’. Then, the operator clicks on the Apply option.

In step 73, the operator passes to the next slide.

In step 74, the operator selects the Custom Animation option from the Slide Show menu. In the ‘Order and Timing’ section's ‘Animation order’ subsection, the operator selects Text1. Then, the operator sets the ‘Start animation’ subsection on the right to ‘Automatically, 0 seconds after previous event’.

In step 75, the operator selects the Slide Transition option from the Slide Show menu. In the panel, the operator sets the ‘Effect’ section at ‘No transition’; the ‘Advance’ section at ‘Automatically after 00:04’; and the ‘Sound’ section at ‘No sound’. Then, the operator clicks on the Apply option.

In step 76, the operator passes to the next slide.

In step 77, the operator selects the Custom Animation option from the Slide Show menu. In the ‘Order and Timing’ section's ‘Animation order’ subsection, the operator selects Text1. Then, the operator sets the ‘Start animation’ subsection on the right to ‘Automatically, 0 seconds after previous event’.

In step 78, in the ‘Order and Timing’ section's ‘Animation order’ subsection, the operator selects Text2. Then, the operator sets the ‘Start animation’ subsection on the right to ‘Automatically, 2.0 seconds after previous event’.

In step 79, in the ‘Order and Timing’ section's ‘Animation order’ subsection, the operator selects Text3. Then, the operator sets the ‘Start animation’ subsection on the right to ‘Automatically, 2.0 seconds after previous event’.

In step 80, in the ‘Order and Timing’ section's ‘Animation order’ subsection, the operator selects Text4. Then, the operator sets the ‘Start animation’ subsection on the right to ‘Automatically, 2.0 seconds after previous event’.

In step 81, in the ‘Order and Timing’ section's ‘Animation order’ subsection, the operator selects Text5. Then, the operator sets the ‘Start animation’ subsection on the right to ‘Automatically, 3.0 seconds after previous event’.

In step 82, the operator selects the Slide Transition option from the Slide Show menu. In the panel, the operator sets the ‘Effect’ section at ‘No transition’; the ‘Advance’ section at ‘Automatically after 00:12.5’; and the ‘Sound’ section at ‘No sound’. Then, the operator clicks on the Apply option.

In step 83, the operator passes to the next slide.

In step 84, the operator selects the Custom Animation option from the Slide Show menu. In the ‘Order and Timing’ section's ‘Animation order’ subsection, the operator selects Text1. Then, the operator sets the ‘Start animation’ subsection on the right to ‘Automatically, 0 seconds after previous event’.

In step 85, in the ‘Order and Timing’ section's ‘Animation order’ subsection, the operator selects Text2. Then, the operator sets the ‘Start animation’ subsection on the right to ‘Automatically, 2.5 seconds after previous event’.

In step 86, in the ‘Order and Timing’ section's ‘Animation order’ subsection, the operator selects Text3. Then, the operator sets the ‘Start animation’ subsection on the right to ‘Automatically, 2.0 seconds after previous event’.

In step 87, in the ‘Order and Timing’ section's ‘Animation order’ subsection, the operator selects Text4. Then, the operator sets the ‘Start animation’ subsection on the right to ‘Automatically, 2.0 seconds after previous event’.

In step 88, in the ‘Order and Timing’ section's ‘Animation order’ subsection, the operator selects Text5. Then, the operator sets the ‘Start animation’ subsection on the right to ‘Automatically, 2.0 seconds after previous event’.

In step 89, in the ‘Order and Timing’ section's ‘Animation order’ subsection, the operator selects Text6. Then, the operator sets the ‘Start animation’ subsection on the right to ‘Automatically, 2.5 seconds after previous event’.

In step 90, the operator selects the Slide Transition option from the Slide Show menu. In the panel, the operator sets the ‘Effect’ section at ‘No transition’; the ‘Advance’ section at ‘Automatically after 00:16’; and the ‘Sound’ section at ‘No sound’. Then, the operator clicks on the Apply option.

In step 91, the operator passes to the next slide.

In step 92, the operator selects the Custom Animation option from the Slide Show menu.

In the ‘Order and Timing’ section's ‘Animation order’ subsection, the operator selects Text1. Then, the operator sets the ‘Start animation’ subsection on the right to ‘Automatically, 0 seconds after previous event’.

In step 93, in the ‘Order and Timing’ section's ‘Animation order’ subsection, the operator selects Text2. Then, the operator sets the ‘Start animation’ subsection on the right to ‘Automatically, 3.0 seconds after previous event’.

In step 94, in the ‘Order and Timing’ section's ‘Animation order’ subsection, the operator selects Text3. Then, the operator sets the ‘Start animation’ subsection on the right to ‘Automatically, 2.0 seconds after previous event’.

In step 95, in the ‘Order and Timing’ section's ‘Animation order’ subsection, the operator selects Text4. Then, the operator sets the ‘Start animation’ subsection on the right to ‘Automatically, 3.0 seconds after previous event’.

In step 96, the operator selects the Slide Transition option from the Slide Show menu. In the panel, the operator sets the ‘Effect’ section at ‘No transition’; the ‘Advance’ section at ‘Automatically after 00:12.5’; and the ‘Sound’ section at ‘No sound’. Then, the operator clicks on the Apply option.

In step 97, the operator passes to the next slide.

In step 98, the operator selects the Custom Animation option from the Slide Show menu. In the ‘Order and Timing’ section's ‘Animation order’ subsection, the operator selects Text1. Then, the operator sets the ‘Start animation’ subsection on the right to ‘Automatically, 0 seconds after previous event’.

In step 99, in the ‘Order and Timing’ section's ‘Animation order’ subsection, the operator selects Text2. Then, the operator sets the ‘Start animation’ subsection on the right to ‘Automatically, 3.0 seconds after previous event’.

In step 100, in the ‘Order and Timing’ section's ‘Animation order’ subsection, the operator selects Text3. Then, the operator sets the ‘Start animation’ subsection on the right to ‘Automatically, 3.0 seconds after previous event’.

In step 101, in the ‘Order and Timing’ section's ‘Animation order’ subsection, the operator selects Text4. Then, the operator sets the ‘Start animation’ subsection on the right to ‘Automatically, 2.5 seconds after previous event’.

In step 102, in the ‘Order and Timing’ section's ‘Animation order’ subsection, the operator selects Text5. Then, the operator sets the ‘Start animation’ subsection on the right to ‘Automatically, 2.0 seconds after previous event’.

In step 103, the operator selects the Slide Transition option from the Slide Show menu. In the panel, the operator sets the ‘Effect’ section at ‘No transition’; the ‘Advance’ section at ‘Automatically after 00:15.5’; and the ‘Sound’ section at ‘No sound’. Then, the operator clicks on the Apply option.

After completion of all the above steps, one can now enjoy the first two paragraphs of the short story “Too much gold” by the great American writer Jack London, as they would appear if published in the ScreenXbook publishing method of the present invention. To do so, one needs to go to the View menu and select the Slide Show option.

“ScreenXbook” Format

It has to be said that the present invention has been inspired by the aura of the cinematographic method of storytelling, and has been made possible and actual by the wide spread of personal computer and Internet.

Though the ingenuity of the two inventions is incomparable, the effect is intended to be very much alike: in the same way as cinematography gave breath of life to painting and photography, the ScreenXbook publishing method is to do the same for books. And in the same way as there are constraints on the time length of a film, the length of screenXbooks should also be limited, and, as a result, would be ideally suited mainly for such printed material as short stories, tales and verses.

The term “printed material” in this application refers to textual and graphic material that can be published in the traditional way on paper. The invention's method comprises electronic publishing of printed material in screenXbook format, whether or not decorated by additional animated/video images and/or audio input.

In order to serve the ultimate purpose of this invention, which is to promote the culture and practice of reading, the “screenXbook” format itself is not being patented. This should allow “home” use of the ScreenXbook publishing method for personal publishing of favorite samples of reading material and personal publishing of classic stories for children to encourage them reading books, and not less importantly would also benefit the artistic community, such as scriptwriters and playwrights, for “pitching” their works.

The “screenXbook” format might also help to those with reading difficulties to assimilate better upon display of their reading/learning material in the “screenXbook” format.

However, public and commercial application of the “ScreenXbook” publishing method, for which this patent application is filed, will be protected to the benefit of the inventor party.

REFERENCES CITED

| | | |
|---|---|---|
| U.S. Pat. No. 5,534,888 | July, 1996 | Blair et al. |
| U.S. Pat. No. 5,761,485 | June, 1998 | Munyan |
| U.S. Pat. No. 5,802,533 | September, 1998 | Walker |
| U.S. Pat. No. 5,860,073 | January, 1999 | Schofield et al. |
| U.S. Pat. No. 5,897,324 | April, 1999 | Tan |
| U.S. Pat. No. 5,991,594 | November, 1999 | Froeber and Kim |
| U.S. Pat. No. 6,105,044 | August, 2000 | Derose and Vogel |
| WO0,225,477 | March, 2002 | Pujol et al. |
| FR2,829,845 | March, 2003 | Schweitzer |

What is claimed is:

1. A method of electronic publishing comprising the following:
a) Input of printed material (i.e. textual and graphic material that can be published in the traditional way on paper) into a computer-code electronic data form which, upon access, displays it for facilitated human reading on screen, as follows:
The text part of the said printed material is displayed on screen in letters of a single light or bright color against a dark-color background, by segments stretching over 1 or 2 horizontal lines/half-lines each;
these segments appear on the screen in the same uniform appearance effect revealing the whole segment, one after the other from top to the bottom, each a number of seconds after the other, at time intervals convenient for their assimilation (and thus filling “screen pages” that follow one another).
The accompanying illustrations that form part of the said printed material are displayed on separate “screen pages”, separately from the main text part.
b) Saving of the said electronic data form into a permanent electronic record form such as file, or recording of the said electronic data form's display onto a cinematographic film or video film.
c) Publishing of the said file by displaying or distributing it on Internet (or another computer network), or by recording it on a media support such as floppy disk or CD.

2. The method of claim 1 wherein the said cinematographic film or video film is publicly displayed through television broadcast or some form of public screening to an audience.

3. The method of claim 1 wherein the said file, following its publishing, is publicly shown to an audience by a means of public display on screen.

4. The method of claim 1 wherein the contents of the said printed material are decorated, during or after publishing, by additional animated/video images and/or audio accompaniment.
